(19) United States
(12) Patent Application Publication
(10) Pub. No.: US 2003/0202948 A1
(43) Pub. Date: Oct. 30, 2003

(54) O/W emulsion

(75) Inventors: Thomas Koini, Saline, MI (US); Gerd H. Dahms, Duisburg (DE)

Correspondence Address: BROOKS & KUSHMAN P.C., 1000 TOWN CENTER, TWENTY-SECOND FLOOR, SOUTHFIELD, MI, 48075, US

(73) Assignee: Wacker-Chemie GmbH, Munich (DE)

(21) Appl. No.: 10/392,600

(22) Filed: Mar. 20, 2003

(30) Foreign Application Priority Data
Apr. 26, 2002 (DE) 102 18 730.4

(51) Int. Cl. A61K 7/42; C07H 15/04

(52) U.S. Cl. 424/59; 536/120

(57) ABSTRACT

O/W emulsions, contain at least one organosilicon compound bearing a glycoside radical and at least one metal oxide are present. The emulsions are stable with respect to pH and exhibit a high degree of dispersion of the metal oxide. The emulsions are particularly useful as cosmetic formulations, in particular as sun screen lotions and creams.

### BACKGROUND OF THE INVENTION

1. Field of the Invention

The invention relates to O/W emulsions and cosmetic preparations based thereon.

2. Background Art

The human skin accustoms itself only slowly to short-wave UV radiation by gradually increasing exposure the solar irradiation up to a certain degree. The harmful effect of higher doses of UV light, especially in the wavelength range between 290 nm and 320 nm, is generally known. Rays in the region of this spectrum defined as UVB region, upon prolonged exposure to light, cause erythema, sunburn or severe burns, depending on the skin type.

However, rays of the UVA spectrum, which is defined as between 320 nm and 400 nm, can also lead to damage to the connective tissue fibers, as a result of which the skin ages prematurely.

To protect against UVA and UVB radiation, numerous organic compounds are known which are able to neutralize or attenuate the harmful UV radiation before it impinges upon the skin. Major disadvantages with the use of these compounds are that they only absorb a small fraction of the light spectrum, exhibit inadequate chemical stability under the action of light, that they can penetrate into the skin, as a result of which allergic and/or hormonal effects are possible (M. Schlumpf, paper, Cosmetic Science Conference at IN-Cosmetics 2001, Düsseldorf, April 2001).

In addition to the organic compounds, inorganic pigments are also used as UV absorbers and/or UV reflectors in cosmetology. The inorganic pigments, which are oxides of titanium, zinc, iron, or aluminum, exhibit very good light protection action over a broad UVA/UVB spectrum based on reflection, scattering and/or absorption of the UV radiation. They are, moreover, chemically stable to the greatest possible extent, i.e. their light protection action is retained at a constant level, no penetration into the skin takes place and no allergic reactions are triggered (Derry, McLaan, Freeman; J. Parenteral & Enteral Nutrition, 7(2), p. 131, 1982). ZnO is recognized, for example in the USA, as a category 1 skin protection agent; see Federal Register, Part II, p. 34641, 1978. A favorable particle size for such pigments is in the range of 100 nm. Applied to the skin, the pigment particles are visually transparent in this size range and exhibit favorable absorption properties. A disadvantage, however, is the difficulty in incorporating and stabilizing such pigments in cosmetic formulations.

If light protection pigments are incorporated into conventional emulsions, then such pigments have a tendency toward considerable agglomerate formation. These agglomerates are in most cases stable in such a way that, upon application, they do not disassociate into their primary particles. A consequence of the poor degree of dispersion manifests itself in an undesired white film formation on the skin (whitening). In addition, the desired UV absorption of the pigment decreases, as a result of which its efficiency as a light protection agent is impaired. A further disadvantage of the agglomerate formation is that they feel sandy when distributed on the skin. (W. Voss, I. Bunge, SPC, Vol. 3, p. 25, 2001).

Furthermore, conventional incorporation of pigments may lead to undesired solubilities of the pigment surface in the water phase. This is particularly so in the case of ZnO, which exhibits pH instability and surface reactivity. Thus, upon contact with acidic/alkaline formulation constituents, Zn ions may be formed or, in reactions with fatty acids, aggregates may arise. Thus, the use of ZnO may lead to the formation of zincates, in which case very often more than 50% of the ZnO pigment can be transferred to the aqueous phase and is thus no longer available for UV absorption. (See Ishii Nobuaki et al., GCI, 2, p. 32, 2001)

In order to solve these agglomeration and reactivity problems, coated pigments are used in every respect. However, the coating is very often not efficient enough to suppress the processes described above.

If it is desired to incorporate micropigments with the finest possible degree of dispersion into O/W emulsions, then lengthy processes are required. For the most part, the pigment has to be predispersed in part of the oil phase used in the emulsion. This dispersion prepared separately from the emulsion is then added subsequently following the preparation of the base emulsion. As a result of the fact that only about 40% of pigment can be incorporated into an oil phase, the overall concentration of UV-ray-absorbing pigment is very limited. At oil component concentrations of 20-30% which are customarily used in sunscreen emulsions, the pigment concentration is accordingly limited to a maximum of 12%.

A further disadvantage of the use of predispersed pigments, is that to achieve an adequate degree of dispersion, it is necessary to add hydrophobic dispersants in an adequate concentration. If the hydrophobic emulsifier used for the predispersion in the O/W sunscreen emulsion to be formulated exceeds a critical concentration, then the hydrophile-lipophile balance (HLB value) shifts into ranges which then leads to an undesired inversion of the O/W emulsion into a W/O emulsion.

### SUMMARY OF THE INVENTION

The object of the present invention is to overcome the disadvantages of the prior art and to provide additives which permit the direct incorporation of high pigment concentrations with conventionally simple preparation processes, where a sufficiently high degree of dispersion of the pigment is achieved and a high stability of the sun protection emulsion is ensured, in particular to overcome the disadvantages of the prior art in order to permit the simple formulation of highly efficient and cosmetically elegant light protection emulsions which are pigment-based. These and other objects are achieved by the invention, wherein O/W emulsions are prepared employing a metal oxide and an organosilicon compound bearing a glycoside radical.

### DETAILED DESCRIPTION OF THE PREFERRED EMBODIMENT(S)

The invention provides an O/W emulsion which comprises at least one organosilicon compound bearing a glycoside radical, and at least one metal oxide.

The metal oxides are preferably the oxides of titanium, zinc, iron, or aluminum, preferably zinc and titanium oxides. They are preferably used in amounts of 0.1-50% by weight, based on the composition, more preferably 1-30% by weight, and most preferably 2-20% by weight. The metal oxides preferably have an average particle size of 5-1000 nm, more preferably 5-400 nm, and most preferably 5-100 nm.

The organosilicon compounds having a glycoside radical are organosilicon compounds having glycoside radicals of units of the formula

$${R}_{a} {R}_{b}^{1} {SiO}_{\frac{4 - a - b}{2}} \qquad ( I )$$

in which

R is identical or different and is a hydrogen atom or organic radical,

a is 0, 1, 2 or 3,

b is 0, 1, 2 or 3 and

R¹ is identical or different and is a radical of the formula

Z—(R²O)_{c}—R³— (II)

in which

Z is a glycoside radical which is made up of 1 to 10, preferably 1 to 4, and more preferably 1 to 2 monosaccharide units,

R² is identical or different and is an alkylene radical,

c is 0 or a number from 1 to 20, preferably 0 or a number from 1 to 15, and more preferably 0 or a number from 1 to 4, and

R³ is an alkylene radical,

with the proviso that the sum of a and b is less than or equal to 3 and the organosilicon compound of units of the formula (I) contains at least one radical R¹ per molecule.

Preferably, the radical R is an optionally substituted hydrocarbon radical having 1 to 18 carbon atoms, where alkyl radicals having 1 to 4 carbon atoms, in particular the methyl radical, are most preferred. Examples of radicals R are alkyl radicals such as the methyl, ethyl, n-propyl, isopropyl, n-butyl, isobutyl, tert-butyl, n-pentyl, isopentyl, neopentyl, and tert-pentyl radicals, hexyl radicals such as the n-hexyl radical, heptyl radicals such as the n-heptyl radical, octyl radicals such as the n-octyl radical and isooctyl radicals such as the 2,2,4-trimethylpentyl radical, nonyl radicals such as the n-nonyl radical, decyl radicals such as the n-decyl radical, dodecyl radicals such as the n-dodecyl radical, and octadecyl radicals such as the n-octadecyl radical; alkenyl radicals, such as the vinyl, allyl, n-5-hexenyl, 4-vinylcyclohexyl and 3-norbornenyl radicals; cycloalkyl radicals such as the cyclopentyl, cyclohexyl, 4-ethylcyclohexyl, cycloheptyl, norbornyl, and methylcyclohexyl radicals; aryl radicals such as the phenyl, biphenylyl, naphthyl, anthryl, and phenanthryl radicals; alkaryl radicals such as the o-, m-, p-tolyl radicals, xylyl radicals, and ethylphenyl radicals; and aralkyl radicals such as the benzyl radical and the α- and the β-phenylethyl radicals.

Examples of monosaccharides of which the glycoside radicals Z may be comprised are hexoses and pentoses, such as glucose, fructose, galactose, mannose, talose, allose, altrose, idose, arabinose, xylose, lyxose and ribose, particular preference being given to glucose.

Examples of alkylene radicals are methylene, ethylene, propylene, butylene, pentylene, hexylene, heptylene, octylene, nonylene, decylene and octadecylene radicals.

The radical R² is preferably the ethylene radical or 1,2-propylene radical, particular preference being given to the ethylene radical.

Preferably, the radical R³ is a linear alkylene radical having 2 to 20 carbon atoms, more preferably linear alkylene radicals having 2 to 8 carbon atoms, in particular the n-propylene radical.

Examples of radicals R¹ are

G—CH₂CH₂CH₂—,

G—(CH₂CH₂O)—CH₂CH₂CH₂—,

G—(CH₂CH₂O)₂—CH₂CH₂CH₂—,

G—(CH₂(CH₃—)CHO)—CH₂CH₂CH₂—,

G—(CH₂(CH₃—)CHO)₂—CH₂CH₂CH₂—,

G—(CH₂CH₂O)—CH₂CH₂(CH₃—)CHCH₂—,

G—(CH₂CH₂O)—CH₂CH₂(CH₃—)CHCH₂—,

where G is a glycoside radical (C₆H₁₁O₆—), and

G₂—CH₂CH₂CH₂—,

G₂—(CH₂CH₂O)—CH₂CH₂CH₂—,

G₂—(CH₂CH₂O)₂—CH₂CH₂CH₂—,

G₂—(CH₂(CH₃—)CHO)—CH₂CH₂CH₂—,

G₂—(CH₂(CH₃—)CHO)₂—CH₂CH₂CH₂—,

G₂—(CH₂CH₂O)—CH₂CH₂(CH₃—)CHCH₂—and

G₂—(CH₂CH₂O)—CH₂CH₂(CH₃—)CHCH₂—,

where G₂ is a glycoside radical made up of two glucose units.

Preferably, radical R¹ is G—CH₂CH₂CH₂—, G—(CH₂CH₂O)—CH₂CH₂CH₂, G₂CH₂CH₂CH₂— or G₂—(CH₂CH₂O)—CH₂CH₂CH₂—, where G—(CH₂CH₂O)—CH₂CH₂CH₂—, and G₂—(CH₂CH₂O)—CH₂CH₂CH₂— are particularly preferred, G is a glucoside radical (C₆H₁₁O₆—) and G₂ is a glycoside radical made up of two glucose units.

Preferably, the organosilicon compounds bearing glycoside radicals according to the invention are those of the formula

R¹ₓR₃₋ₓSiO—[(SiRR¹O)ₘ—(SiR₂O)ₙ]_{y}—SiR₃₋ₓR¹ₓ (III),

in which R and R₁ have the meanings given above,

m is identical or different and is 0 or a number from 1 to 200, preferably 0 or a number from 1 to 100, more preferably 0 or a number from 1 to 50,

n is identical or different and is 0 or a number from 1 to 1000, preferably 0 or a number from 1 to 500, more preferably 0 or a number from 1 to 100,

x is 0 or 1 and

y is 0 or a number from 1 to 1200, preferably 0 or a number from 1 to 600, more preferably 0 or a number from 1 to 100,

with the proviso that the compound of the formula (III) has at least one radical R¹.

If, in the organosilicon compounds bearing glycoside radicals according to formula (III), m is, on average, different from 0, x is preferably 0. If, in the organosilicon compounds having glycoside radicals according to formula (III), x is, on average, different from 0, m is preferably 0.

Although not shown by formula (III), it is possible for up to 10 mol % of the diorganosiloxane units to be replaced by other siloxane units, such as, for example, RSiO_{3/2}—, R¹SiO_{3/2} and/or SiO_{4/2} units, where R and R¹ have the meanings given above.

The organosilicon compounds bearing glycoside radicals according to the invention can be prepared by various methods, which are described in DE 43 06 041 A1 or U.S. Pat. No. 5,831,080. Further suitable organosilicon compounds bearing glycoside radicals are described in EP 1004614A1 (Wacker-Chemie GmbH), EP 0879840, JP5186596 and are hereby incorporated by reference. By “bearing a glycoside radical” is meant that at least one glycoside radical is present.

The organosilicon compounds bearing glycoside radicals according to the invention are preferably used in amounts of from 0.1 to 70% by weight, based on the metal oxide concentration used, more preferably between 0.2 and 50% by weight, and most preferably between 1 and 20% by weight, depending on the metal oxide concentration used. The organosilicon compounds bearing glycoside radicals according to the invention are preferably dissolved in an oil.

It is most surprising that equally good results are achieved irrespective of the ionic character of the emulsifiers used for the stabilization of the O/W emulsion. Even variation of the oil polarity of the oils used in the O/W emulsion does not lead to any losses in the efficiency of the organosilicon compounds bearing glycoside radicals which are used. Suitable oils are silicone oils and derivatives thereof, e.g. high- and low-viscosity organopolysiloxanes such as dimethylpolysiloxanes, methylphenylpolysiloxanes, methylhydrogenpolysiloxanes and dimethylsiloxane-methylphenylsiloxane copolymers; cyclic siloxanes such as octamethylcyclotetrasiloxane, decamethylcyclopentasiloxane, dodecamethylcyclo-hexasiloxane, and tetramethyltetrahydrogencyclotetrasiloxanes; silicone rubbers such as dimethylpolysiloxanes and dimethylsiloxane-methylphenylsiloxane copolymers with a high degree of polymerization, and cyclosiloxane solutions of silicone rubber, trimethylsiloxysilicate, cyclosiloxane solutions of trimethylsiloxysilicate, higher alkoxy-modified silicones such as stearoxysilicones, higher fatty acid-modified silicones, alkyl-modified silicones, amino-modified silicones, fluoro-modified silicones, and silicone resin solutions. The aforementioned silicones are not structurally restricted, but can have any straight-chain, branched, crosslinked and cyclic structure.

Additional oils include oils/waxes of vegetable and animal origin, e.g. avocado oil, linseed oil, almond oil, carnauba wax, liver oil, candelilla wax, tallow derivatives, apricot oil, hydrogenated oil, wheatgerm oil, sesame oil, ricegerm oil, sugar cane wax, jojoba oil, soybean oil, tea oil, fat, rape oil, palm oil, castor oil, sunflower oil, jojoba wax, coconut oil, fatty acid glycerides, hydrogenated oils, peanut oil, lanolin and derivatives, and hexyl laurate; ester oils, e.g. diisobutyl adipate, 2-hexyldecyl adipate, di-2-heptylundecyl adipate, N-alkylglycol monoisostearate, isocetyl isostearate, trimethylolpropane triisostearic acid, ethylene glycol, di-2-ethylhexanoic esters, cetyl-2-ethyl hexanoate, trimethylolpropane tri-2-ethylhexanoic esters, pentaerythritol tetra-2-ethylhexanoic esters, cetyl octanoate, octyldodecyl gum esters, oleyl oleate, octyldodecyl oleate, decyl oleate, neopentyl glycol dicaprylic esters, triethyl citrate, 2-ethylhexyl cinnamate, amyl acetate, ethyl acetate, butyl acetate, isocetyl stearate, butyl stearate, diisopropyl sebacate, di-2-ethylhexyl sebacate, cetyl lactate, myristyl lactate, isopropyl palmitate, 2-ethylhexyl palmitate, 2-hexyldecyl palmitate, 2-heptylundecyl palmitate, cholesteryl-12 hydroxystearate, dipentaerythritol fatty acid esters, isopropyl myristate, octyldodecyl myristate, 2-hexyldecyl myristate, myristyl myristate, hexyldecyl dimethyloctanoate, ethyl laurate, hexyl laurate, N-lauroyl-L-glutamic acid, 2-octyldodecyl esters and diisostearylmaleic acid; liquid fatty alcohols, e.g. lauryl alcohol, myristyl alcohol, palmityl alcohol, stearyl alcohol, behenyl alcohol, hexadecyl alcohol, oleyl alcohol, isostearyl alcohol, octadodecanol, cetylstearyl alcohol, 2-decyltetradecinol, cholesterol, phytosterol, monostearyl glycerol ether (batyl alcohol), and monooleyl glyceryl ether (ceryl alcohol); paraffin-based oils/waxes, e.g. ozokerite, squalane, ceresine, paraffin, paraffin wax, liquid paraffin, pristane, polyisobutylene, microcrystalline wax and vaseline; higher fatty acids, e.g. lauric acid, stearic acid, behenic acid, undecanoic acid, oleic acid, linoleic acid, eicosapentanoic acid (EPA), docosahexanoic acid (DHA), and isostearic acid. As oils, preference is given to cyclomethicones, hexamethyldisiloxane and dimethicones, and particular preference is given to cyclohexasiloxane, cyclopentasiloxane, cyclotetrasiloxane and low-viscosity dimethicones. These oils can be used in any mixtures with one another.

The oils are preferably used in the ratio 9 parts by weight of oil to 1 part by weight of organosilicon compounds bearing glycoside radicals, more preferably in the ratio 7 parts by weight of oil to 1 part by weight of organosilicon compounds bearing glycoside radicals, in the ratio 2 parts by weight of oil to 1 part by weight of organosilicon compounds bearing glycoside radicals, and most preferably in the ratio 4 parts by weight of oil to 1 part by weight of organosilicon compounds bearing glycoside radicals.

The quality of the degree of dispersion achieved in the emulsion is not impaired by conventional organic UV absorbers, such as those of the benzoic acid type, anthranyl acid type, salicyl acid type, succinic acid type, benzophenone type, uranyl acid type, dibenzoyl acid type, or cinnamyl acid type. All these organic UV absorbers are well known to those skilled in the art.

It has also been surprisingly discovered that modifications of the water phase do not impair the efficiency of the O/W emulsion which comprises organosilicon compounds bearing glycoside radicals together with micropigments.

To prepare a formulation, it is further possible to use ingredients which are normally used for cosmetic preparations, such as water, film formers, oil-soluble compounds, resins, UV absorbers, moisturizers, antiseptic agents, preservatives, perfume, salts, antioxidants, pH regulators, complexing agents, anti-inflammatory agents, skin-improving agents (e.g. skin-bleaching agents, cell activators, improvers for rough and dry skin, stimulators of blood circulation), vitamins, amino acids, nucleic acids, hormones, etc., provided they do not exhibit any adverse influences on the desired effects.

Emulsifiers may be anionic, cationic, nonionic and amphoteric in nature. Examples of anionic emulsifiers which can be used include saponified fatty acids, e.g. sodium stearate/triethanolamine palmitate, alkyl ether carboxylic acids and their salts, salts of amino acid-fatty acid condensates, alkanesulfonates, alkenesulfonates, sulfonated fatty acid esters, alkyl sulfates, sulfates of higher secondary alcohols, alkyl and aryl ether sulfates, fatty acid ether sulfates, fatty acid alkylamide sulfates, ether sulfates, alkyl phosphates, ether phosphates, alkyl aryl ether phosphates, amide phosphates, and emulsifiers of the N-acylamino acid type.

Examples of cationic emulsifiers which can be used include amine salts, e.g. alkylamine salts, polyamines and nonalcoholic aminic fatty acid derivatives, quaternary alkylammonium salts, and quaternary arylammonium salts.

Examples of nonionic emulsifiers which can be used include sorbitan fatty acid esters, glycerol fatty acid esters, polyglycerol fatty acid esters, propylene glycol fatty acid esters, polyethylene glycol fatty acid esters, polyoxyethylene alkyl ethers, polyoxypropylene alkyl ethers, polyoxyethylene alkyl phenyl ethers, polyoxyethylene fatty acid esters, polyoxyethylene sorbitan fatty acid esters, polyoxyethylene sorbitol fatty acid esters, polyoxyethylene glycerol fatty acid esters, polyoxyethylene propylene glycol fatty acid esters, polyoxyethylenes of castor oil and hydrogenated castor oil, polyoxyethylene phytostanol ethers, polyoxyethylene phytosterol ethers, polyoxyethylene cholestanol ethers, polyoxyethylene cholesterol esters, polyoxyalkylene-modified organopolysiloxanes, organopolysiloxanes modified with polyoxyalkyl and alkyl groups, alkanolamides, sugar ethers and sugar amides, and fatty alcohols.

Examples of amphoteric emulsifiers which can be used include betaine aminocarboxylates and imidazole derivatives.

As emulsifiers, preference is given to sorbitan fatty acid esters, glycerol fatty acid esters, polyglycerol fatty acid esters, propylene glycol fatty acid esters, polyethylene glycol fatty acid esters, polyoxyethylene alkyl ethers, polyoxypropylene alkyl ethers, polyoxyethylene sorbitan fatty acid esters, polyoxyethylene sorbitol fatty acid esters, polyoxyethylenes glycerol fatty acid esters, polyoxyethylenepropyleneglycol fatty acid esters, polyoxyethylated hydrogenated castor oil, polyoxyalkylene-modified organopolysiloxanes, organopolysiloxanes modified with polyoxyalkyl and alkyl groups, fatty alcohols, most preferably alkyl ether carboxylic acids and their salts, alkyl sulfates, glycerol fatty acid esters, polyethylene glycol fatty acid esters, sucrose fatty acid esters, polyoxyalkylene-modified organopolysiloxanes, organopolysiloxanes modified with polyoxyalkyl and alkyl groups, and fatty alcohols.

The invention further provides a method of preparing the inventive O/W emulsions, where an O/W emulsion is prepared to which, at a temperature of less than 50° C., the organosilicon compound bearing a glycoside radical, dissolved in a solvent, is added with the metal oxide.

The method of preparing the O/W emulsions in which the micropigment to be used is added together with the organosilicon compound having a glycoside radical (dissolved in solvent) involve addition after cooling the emulsion to a temperature of less than 50° C. In this method, particular preference is given to the addition in the range from 20 to 45° C.

The invention further provides a cosmetic preparation based on the O/W emulsion according to the invention. The cosmetic preparation is preferably prepared as lotion or cream. This cosmetic preparation is particularly suitable as a sun protection composition. The invention further provides for the use of the cosmetic preparation for sun protection.

The invention may be illustrated with respect to formulations containing ZnO. By the use of only 1% of organosilicon compound bearing glycoside radicals, predissolved in solvent, the troublefree incorporation of 20% of ZnO into a conventionally prepared O/W guide formulation if possible. If use of the organosilicon compound bearing a glycoside radical is omitted, the O/W formulations invert at a use concentration of just 10% of ZnO to give W/O emulsions.

At high pigment concentration, and without being bound thereby, it is believed that the phase inversion which is brought about by a critical concentration of 10% of ZnO in conventional dispersions can be explained as follows. As ZnO is added to the emulsion, the pigment interacts with the emulsifier used. In this process, the hydrophilic surfactants which are responsible for stabilizing the O/W emulsion are adsorbed to the strongly polar ZnO surface. The amount of adsorbed hydrophilic surfactant depends on the concentration of the ZnO pigment used. In other words, as the zinc oxide concentration increases, the content of hydrophilic surfactant in the O/W emulsion decreases. As a result, the HLB value of the emulsifier mixture used likewise decreases. As soon as the HLB value drops below 6, the system has a tendency to invert to give a W/O emulsion.

If, however, the zinc oxide powder is incorporated together with the organosilicon compound having a glycoside radical into the emulsion, the organosilicon compound bearing a glycoside radical surprisingly attaches more rapidly to the ZnO surface, despite its high molecular weight and its relatively low hydrophilicity, than the low molecular weight hydrophilic emulsifiers which are used in O/W emulsion systems. As a result of the high affinity of the organosilicon compound bearing a glycoside radical to the pigment surface, the latter is shielded almost completely.

With respect to pH of the compositions, as a result of this good shielding effect described above, the undesired formation of zincate is also almost completely suppressed. Detection of alkaline-reacting zincates which form at the ZnO surface can be carried out using pH measurement. A comparison, as shown in table 1, of the pH values of the emulsions both with and without organosilicon compound bearing a glycoside radical clearly shows an increase in the pH with increasing ZnO concentration in the emulsion without organosilicon compound bearing a glycoside radical while the increase in the pH in the case of the emulsion with organosilicon compound bearing a glycoside radical is barely noticeable.

| | pH of emulsion with | pH of emulsion without |
|---|---|---|
| ZnO concentration | OCbeG (predissolved in | OCbeG (predissolved in |
| (% by wt.) | solvent) | solvent) |
| 0 | 5.6 | 5.4 |
| 3 | 5.7 | 6.9 |
| 5 | 5.8 | 7 |
| 10 | 5.8 | 7.2 |
| 15 | 5.9 | 7.4 |
| 20 | 6 | * |

*Emulsion inverted
OCbeG = organosilicon compound bearing a glycoside radical

The considerable formation of zincate in the emulsion without the organosilicon compound bearing a glycoside radical has an autocatalytic effect due to the increased pH values which arise in the process, i.e. more and more zinc oxide is converted into zincate with increasing storage period. This operation too can be monitored by means of pH measurements. If the pH of the emulsion containing 5% of ZnO which does not comprise an organosilicon compound bearing a glycoside radical is observed, it is established that the pH increases considerably over the storage time, while in the case of the emulsion with organosilicon compound having a glycoside radical, no pH increase is observed.

| | pH of emulsion with | pH of emulsion without |
|---|---|---|
| | OCbeG (predissolved | OCbeG (predissolved |
| Time (days) | in solvent) | in solvent) |
| 0 | 5.8 | 7 |
| 1 | 5.8 | 7.9 |
| 3 | 5.9 | 8.3 |
| 7 | 5.9 | 8.4 |
| 14 | 5.9 | 8.6 |

OCbeG = organosilicon compound bearing a glycoside radical

The pH analysis clearly shows how the ZnO pigment concentration for the emulsion without organosilicon compound bearing a glycoside radical decreases over time. In a light protection emulsion, this results in the light protection effect of such an emulsion likewise decreasing with increasing storage period. In addition to the undesired decrease in the pigment concentration, the high pH may also lead to skin irritations.

The addition of organosilicon compound having a glycoside radical according to the invention also leads to a considerably better degree of dispersion of the micropigment ZnO, which, as is known, can bring about a significant increase in the light protection action by up to 50% (DE 195 48 015 A1). In addition to the increase in the light protection action, the use of organosilicon compound bearing a glycoside radical leads to a significant improvement in the cosmetic properties of emulsions based on micropigments. Thus, an emulsion containing 20% by weight of ZnO which contains 1.5% by weight of organosilicon compound bearing a glycoside radical does not show any kind of white film formation even on “Mediterranean” skin, e.g. skin of olive complexion.

If TiO₂ is exchanged for ZnO, the positive properties of the organosilicon compound bearing a glycoside radical are maintained. Due to the low transparency of the TiO₂ micropigment, the maximum concentration of the TiO₂ pigment which leads to no visible white film formation following spreading, even on Mediterranean skin, is lower. Surprising, however, is that at the very high concentration of 12.5% by weight of TiO₂, transparent films are still formed. The surprisingly high transparency of the micropigment films formed on the skin is to be attributed firstly to the high degree of dispersion of the micropigments, and secondly to the stability of the micropigment dispersion when the light protection emulsion is rubbed onto the skin. The surprisingly high degree of dispersion of the TiO₂ pigment can be demonstrated both via microscopic analysis and via determination of the particle size by means of laser light scattering. If the laser light scattering method is used, for the inventive micropigment distribution in a customary oil-in-water emulsion, an average particle diameter of ˜200 nm is achieved even with a low to moderate input of energy, compared with standard commercial TiO₂ dispersions, the average diameter of which is in the range between 350 and 400 nm. The high affinity of the organosilicon compound bearing a glycoside radical to the TiO₂ particles guarantees that the dispersion is not adversely affected even under the shear forces generated as the emulsion is rubbed onto the skin. This leads to a homogeneous film formation on the skin which guarantees a high light protection efficiency. Furthermore, as a result of the shear stability when the light protection emulsion is rubbed onto the skin, the very good degree of dispersion of the TiO₂ is not adversely affected, and thus an above average transparency is achieved.

Even at an extremely high concentration of TiO₂ of more than 10% by weight, in the case of use of organosilicon compound bearing a glycoside radical the sandy feel upon application to the skin which is often perceived as being unpleasant at use concentrations as low as 3% by weight of TiO₂ is not experienced.

The high affinity of the organosilicon compound bearing a glycoside radical also brings about the formation of a coherent protective film on inadequately coated TiO₂. It is known that especially microfine TiO₂, being a semiconductor under the action of light, represents a strong oxidizing agent toward organic substances. The resulting oxidation products bring about a pH decrease which is not observed in TiO₂-containing emulsions with organosilicon compounds bearing a glycoside radical.

| Example | 1 | 2 |
|---|---|---|
| Phase A | | |
| Polyglyceryl-3 methyl glucose isostearate | 4 | 4 |
| Octyl stearate | 8 | 8 |
| Cetearyl isononanoate | 3 | 3 |
| Isoamyl p-methoxycinnamate | 9 | 9 |
| Tocopherol acetate | 1 | 1 |
| Butyl methoxydibenzoylmethane | 0.5 | 0.5 |
| Phase B | | |
| Water | 63.7 | 59.7 |
| Glycerol | 5 | 5 |
| Xanthan gum | 0.3 | 0.3 |
| Phase C | | |
| 20% OCbeG in cyclomethicone | 0 | 4 |
| Titanium dioxide | 4 | 4 |
| Phase D | | |
| Preservative | q.s. | q.s. |
| | 100 | 100 |

OCbeG = organosilicon compound bearing a glycoside radical

| | pH of emulsion 1 | pH of emulsion 2 with |
|---|---|---|
| | without OCbeG | OCbeG (predissolved in |
| Time (days) | (predissolved in solvent) | solvent) |
| 1 | 7 | 7 |
| 10 | 6.5 | 6.9 |
| 36 | 5.6 | 6.7 |
| 150 | 5.2 | 6.7 |

OCbeG = organosilicon compound bearing a glycoside radical

In order to achieve homogeneous distribution of the micropigment, the base emulsion consisting of the oil phase A and the water phase B is firstly prepared. For this, the two phases are heated separately to 75° C. with stirring until the two phases are homogeneously mixed. Then, oil phase A is emulsified into water phase B with continuous stirring, and the emulsion is homogenized with a rotor stator stirrer. The resulting emulsion is then cooled to 40° C. with stirring. The stirring operation is interrupted at this point. Now, firstly the pigment and then the organosilicon compound bearing a glycoside radical, is added to the emulsion. The whole mixture is then homogenized again with a rotor stator or high-speed propeller stirrer until an optimum pigment distribution is achieved. Assessment of the pigment distribution is carried out by means of microscopy using polarized light. After an optimum pigment distribution has been reached, the emulsion is cooled to 25° C. with stirring.

To ensure optimum process control in the case of industrially prepared emulsions, the experiments were carried out in a 2 kg processing plant from IKA LA 2000 V, the construction of which corresponds to that of plants used in industry.

Pigment-containing Sun Protection Emulsions Without Organic Filters

Zinc Oxide

| Example 1 | A |
|---|---|
| Phase A | |
| Glycerol stearate, cetearyl alcohol, sodium stearoyl lactylate | 5 |
| (BioBase S) | |
| Laureth-23 | 1 |
| Cetearyl isononanoate | 8 |
| Heptamethylnonane | 5 |
| Tocopherol acetate | 1 |
| Phase B | |
| Water | 44.2 |
| Glycerol | 5 |
| Xanthan gum | 0.3 |
| Phase C | |
| 10% OCbeG in cyclomethicone | 10 |
| Zinc oxide | 20 |
| Phase D | |
| Preservative | q.s. |
| | 100 |

OCbeG = organosilicon compound bearing a glycoside radical

| Example 2 | B |
|---|---|
| Phase A | |
| Glycerol stearate, cetearyl alcohol, sodium stearoyl lactylate | 5 |
| (BioBase S) | |
| Laureth-23 | 1 |
| Tridecyl salicylate | 8 |
| Capric/caprylic triglycerides | 5 |
| Tocopherol acetate | 1 |
| Phase B | |
| Water | 44.2 |
| Glycerol | 5 |
| Xanthan gum | 0.3 |
| Phase C | |
| 10% OCbeG in cyclomethicone | 10 |
| Zinc oxide | 20 |
| Phase D | |
| Preservative | q.s. |
| | 100 |

OCbeG = organosilicon compound bearing a glycoside radical

| Example 3 | C |
|---|---|
| Phase A | |
| Glycerol stearate, PEG-100 stearate (Arlacel 165) | 1 |
| Glyceryl stearate | 2 |
| Cetearyl alcohol | 2 |
| Laureth-23 | 1 |
| Cetearyl isononanoate | 8 |
| Heptamethylnonane | 5 |
| Tocopherol acetate | 1 |
| Phase B | |
| Water | 54.2 |
| Glycerol | 5 |
| Xanthan gum | 0.3 |
| Phase C | |
| 10% OCbeG in cyclomethicone | 10 |
| Zinc oxide | 10 |
| Phase D | |
| Preservative | q.s. |
| | 100 |

| Example 4 | D |
|---|---|
| Phase A | |
| Glycerol stearate, cetearyl alcohol, sodium stearoyl lactylate | 5 |
| (BioBase S) | |
| Laureth-23 | 1 |
| Glyceryl stearate, PEG-100 stearate (Arlacel 165) | 0 |
| Glyceryl stearate | 0 |
| Cetearyl alcohol | 0 |
| Cetearyl isononanoate | 8 |
| Heptamethylnonane | 5 |
| Tocopherol acetate | 1 |
| Phase B | |
| Water | 54.2 |
| Glycerol | 5 |
| Xanthan gum | 0.3 |
| Phase C | |
| 10% OCbeG in cyclomethicone | 10 |
| Zinc oxide | 10 |
| Phase D | |
| Preservative | q.s. |
| | 100 |

OCbeG = organosilicon compound bearing a glycoside radical

Titanium Dioxide

| Example 5 | E |
|---|---|
| Phase A | |
| Glycerol stearate, cetearyl alcohol, sodium stearoyl lactylate | 5 |
| (BioBase S) | |
| Laureth-23 | 1 |
| Cetearyl isononanoate | 8 |
| Heptamethylnonane | 5 |
| Tocopherol acetate | 1 |
| Phase B | |
| Water | 44.2 |
| Glycerol | 5 |
| Xanthan gum | 0.3 |
| Phase C | |
| 10% OCbeG in cyclomethicone | 10 |
| Zinc oxide | 15 |
| Phase D | |
| Preservative | q.s. |
| | 100 |

OCbeG = organosilicon compound bearing a glycoside radical

| Example 6 | F |
|---|---|
| Phase A | |
| Glycerol stearate, cetearyl alcohol, sodium stearoyl lactylate | 5 |
| (BioBase S) | |
| Laureth-23 | 1 |
| Tridecyl salicylate | 8 |
| Capric/caprylic triglycerides | 5 |
| Tocopherol acetate | 1 |
| Phase B | |
| Water | 44.2 |
| Glycerol | 5 |
| Xanthan gum | 0.3 |
| Phase C | |
| 10% OCbeG in cyclomethicone | 10 |
| Titanium dioxide | 15 |
| Phase D | |
| Preservative | q.s. |
| | 100 |

OCbeG = organosilicon compound bearing a glycoside radical

| Example 7 | G |
|---|---|
| Phase A | |
| Glyceryl stearate, PEG-100 stearate (Arlacel 165) | 1 |
| Glyceryl stearate | 2 |
| Cetearyl alcohol | 2 |
| Laureth-23 | 1 |
| Cetearyl isononanoate | 8 |
| Heptamethylnonane | 5 |
| Tocopherol acetate | 1 |
| Phase B | |
| Water | 44.2 |
| Glycerol | 5 |
| Xanthan gum | 0.3 |
| Phase C | |
| 10% OCbeG in cyclomethicone | 10 |
| Titanium dioxide | 15 |
| Phase D | |
| Preservative | q.s. |
| | 100 |

| Example 8 | H |
|---|---|
| Phase A | |
| Glyceryl stearate, PEG-100 stearate (Arlacel 165) | 1 |
| Glyceryl stearate | 2 |
| Cetearyl alcohol | 2 |
| Laureth-23 | 1 |
| Tridecyl salicylate | 8 |
| Capric/caprylic triglyceride | 5 |
| Tocopherol acetate | 1 |
| Phase B | |
| Water | 44.2 |
| Glycerol | 5 |
| Xanthan gum | 0.3 |
| Phase C | |
| 10% OCbeG in cyclomethicone | 10 |
| Titanium dioxide | 15 |
| Phase D | |
| Preservative | q.s. |
| | 100 |

OCbeG = organosilicon compound bearing a glycoside radical

Pigment-containing Sun Protection Formulations with Organic Filters

Zinc Oxide

| Example 9 | A |
|---|---|
| Phase A | |
| Glycerol stearate, cetearyl alcohol, sodium stearoyl lactylate | 5 |
| (BioBase S) | |
| Laureth-23 | 1 |
| Octyl methoxycinnamate | 10 |
| Cetearyl isononanoate | 8 |
| Heptamethylnonane | 5 |
| Tocopherol acetate | 1 |
| Phase B | |
| Water | 34.2 |
| Glycerol | 5 |
| Xanthan gum | 0.3 |
| Phase C | |
| 10% OCbeG in cyclomethicone | 10 |
| Zinc oxide | 20 |
| Phase D | |
| Preservative | q.s. |
| | 100 |

OCbeG = organosilicon compound bearing a glycoside radical

Titanium Dioxide

| Example 10 | F |
|---|---|
| Phase A | |
| Glycerol stearate, cetearyl alcohol, sodium stearoyl lactylate | 5 |
| (BioBase S) | |
| Sodium stearyl lactylate | 2.5 |
| Tridecyl salicylate | 10 |
| Cetearyl isononanoate | 8 |
| Heptamethylnonane | 5 |
| 4-methylbenzylidenecamphor | 3 |
| Octocrylene | 3 |
| Tocopherol acetate | 1 |
| Phase B | |
| Water | 41.3 |
| Glycerol | 5 |
| Xanthan gum | 0.5 |
| Alkyl acrylates/C10-30 alkyl acrylate crosspolymer | 0.2 |
| Phase C | |
| 10% OCbeG in cyclomethicone | 10 |
| Titanium dioxide | 5 |
| Phase D | |
| Preservative | q.s. |
| | 100 |

OCbeG = organosilicon compound bearing a glycoside radical

| Example 11 | G |
|---|---|
| Phase A | |
| Glycerol stearate, cetearyl alcohol, sodium stearoyl lactylate | 5 |
| (BioBase S) | |
| Laureth-23 | 1 |
| Octyl methoxycinnamate | 10 |
| Cetearyl isononanoate | 8 |
| Heptamethylnonane | 5 |
| Tocopherol acetate | 1 |
| Phase B | |
| Water | 39.7 |
| Glycerol | 5 |
| Xanthan gum | 0.3 |
| Phase C | |
| 10% OCbeG in cyclomethicone | 10 |
| Titanium dioxide | 15 |
| Phase D | |
| Preservative | q.s. |
| | 100 |

OCbeG = organosilicon compound bearing a glycoside radical.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. An O/W emulsion comprising at least one organosilicon compound bearing a glycoside radical and at least one metal oxide.

2. The O/W emulsion of claim 1, wherein the organosilicon compound bearing a glycoside radical is organosilicon compounds having glycoside radicals of units of the formula
$${R}_{a} {R}_{b}^{1} {SiO}_{\frac{4 - a - b}{2}} \qquad ( I )$$
in which
R is identical or different and is a hydrogen atom or organic radical,
a is 0, 1, 2 or 3,
b is 0, 1, 2 or 3 and
R¹ is identical or different and is a radical of the formula
Z—(R²O)_{c}—R³— (II)
in which
Z is a glycoside radical which is made up of 1 to 10 monosaccharide units,
R² is identical or different and is an alkylene radical,
c is 0 or a number from 1 to 20 and
R³ is an alkylene radical,
with the proviso that the sum of a and b is less than or equal to 3 and the organosilicon compound of units of the formula (I) contains at least one radical R¹ per molecule.

3. The O/W emulsion of claim 2, wherein, Z is a glycoside radical which comprises 1 to 2 monosaccharide units.

4. The O/W emulsion of claim 2, wherein the organosilicon compound bearing a glycoside radical comprises one of the formula
R¹ₓR₃₋ₓSiO—[(SiRR¹O)ₘ—(SiR₂O)ₙ]_{y}—SiR₃₋ₓR¹ₓ (III),
in which R and R¹ have the meanings given in claim 1,
m is identical or different and is 0 or a number from 1 to 200,
n is identical or different and is 0 or a number from 1 to 1000,
x is 0 or 1, and
y is 0 or a number from 1 to 1200,
with the proviso that the compound of the formula (III) has at least one radical R¹.

5. The O/W emulsion of in claim 4, wherein when m is on average different from 0, x is 0.

6. The O/W emulsion of claim 4, wherein when x is on average different from 0, m is 0.

7. The O/W emulsion of claim 1, wherein the metal oxide is one or more selected from the group consisting of the oxides of titanium, zinc, iron, and aluminum.

8. The O/W emulsion of claim 2, wherein the metal oxide is one or more selected from the group consisting of the oxides of titanium, zinc, iron, and aluminum, and said metal oxide is present in the form of particles having a mean particle size of less than 1 μm.

9. The O/W emulsion of claim 4, wherein the metal oxide is one or more selected from the group consisting of the oxides of titanium, zinc, iron and aluminum.

10. A method for preparing the O/W emulsion of claim 1, which comprises preparing a base O/W emulsion to which, at a temperature of less than 50° C., the organosilicon compound bearing a glycoside radical and dissolved in a solvent is added with the metal oxide.

11. A cosmetic preparation, comprising the O/W emulsion of claim 1.

12. The cosmetic preparation as claimed in claim 11, which is in the form of a lotion or cream.

13. The cosmetic preparation of claim 11 which is a sun screen preparation, in which metal oxide particles having a mean particle size in the range of 5 nm to 400 nm are present.

14. A method of protecting the skin from the effects of solar UV radiation, comprising applying the cosmetic preparation of claim 1 to the skin.

15. A method of protecting the skin from the effects of solar UV radiation, comprising applying the cosmetic preparation of claim 11 to the skin.

16. A method of protecting the skin from the effects of solar UV radiation, comprising applying the cosmetic preparation of claim 12 to the skin.

17. A method of protecting the skin from the effects of solar UV radiation, comprising applying the cosmetic preparation of claim 13 to the skin.
